(19) United States
(12) Patent Application Publication
(10) Pub. No.: US 2021/0113390 A1
(43) Pub. Date: Apr. 22, 2021

(54) Reinforced Fastening Tabs

(71) Applicant: 3M INNOVATIVE PROPERTIES COMPANY, St. Paul, MN (US)

(72) Inventors: Thomas J. Gilbert, St. Paul, MN (US); Michael R. Gorman, Woodbury, MN (US); Ryan M. Luepke, Eagan, MN (US)

(21) Appl. No.: 16/965,327

(22) PCT Filed: Feb. 7, 2019
(86) PCT No.: PCT/IB2019/051003

§ 371 Date: Jul. 28, 2020

Related U.S. Application Data
(60) Provisional application No. 62/628,462, filed on Feb. 9, 2018.

(51) Int. Cl. A61F 13/62; A61F 13/49; A61F 13/56

(52) U.S. Cl. CPC A61F 13/622; A61F 13/5622; A61F 13/49007

(57) ABSTRACT

A composite article for use in, e.g., a disposable diaper including a strip of hook material tab where the strip extends into the part of the tab used for attachment to, e.g., the “ear” which conventionally extends to each side of a disposable diaper's chassis. Such an arrangement increases the tear strength of the tab, providing more secure attachment in the face of movement by the wearer.

### TECHNICAL FIELD

The present disclosure relates generally to the formation of tabs useful for, e.g., the manufacture of disposable diapers.

### BACKGROUND

Hook-and-loop fasteners are widely used as garment fasteners and for securing disposable diapers and feminine hygiene products. For example, in the manufacture of disposable diapers it is known to provide a pair of tabs each bearing a portion of hook material, the tabs extending from laterally extending ears on the back of the diaper. The diaper is disposed under the crotch of the wearer and these tabs are pressed against a landing zone having loop material thereon so as to form a wearable garment.

One of the most important design considerations for the tab is to provide sufficient tensile and tear strength such that the tab does not break during the diapering process. Ideally this carrier would be very soft and very inexpensive, but since it must have this strength there are some limitations to what types of materials can be used. For example, there might be a desire to mount the tab material on a nonwoven carrier with low basis weight, but since this type of material has low tensile and tear strength, it would not function in use. One solution to address this today is to increase the basis weight of the carrier or add a strengthening layer to the carrier to give it sufficient strength, but this adds cost and complexity.

### SUMMARY

The present disclosure provides a composite article for use in, e.g., a disposable diaper including a strip of hook material tab where the strip extends into the part of the tab used for attachment to, e.g., the “ear” which conventionally extends to each side of a disposable diaper's chassis. Such an arrangement increases the tensile and tear strength of the tab, providing more secure attachment in the face of movement by the wearer. In one aspect, the present disclosure provides a composite article comprising a layer and a strip having raised hooks on a first side, wherein a portion of the first side is bonded to the layer. The raised hooks may be crushed before accomplishing this bonding by, e.g., thermal or ultrasonic bonding. Alternatively, the portion which is bonded may have adhesive sufficient to submerge or partially submerge the raised hooks. The layer will often be the chassis of a disposable diaper or catamenial article. In some convenient embodiments, the layer will be a portion of the chassis that extends to the side, forming an “ear.” The strip may be reinforced by a support fabric, and a layer carrying indicia may be disposed between the strip and the support fabric. In some convenient embodiments, the support fabric may comprise loop material.

### LISTING OF EXEMPLARY EMBODIMENTS

Embodiment A. A composite article comprising a layer and a strip having raised hooks on a first side, wherein a portion of the first side is bonded to the layer.

Embodiment B. The composite article according to Embodiment A wherein the layer is a portion of a disposable diaper.

Embodiment C. The composite article according to Embodiment A, wherein the strip has a second side opposite the first side, and wherein the second side is bonded to a support fabric.

Embodiment D. The composite article according to Embodiment A, wherein the support fabric comprises loop material.

Embodiment E. The composite article according to Embodiments A or D, wherein the strip is bonded to the layer at a proximal end of the strip, and wherein a tab at the distal end of the strip is free of raised hooks.

Embodiment F. The composite article according to Embodiment E wherein the distal end has flattened hooks.

Embodiment G. The composite article according to any of the previous Embodiments wherein the basis weight of the strip is less than 80 g/m².

Embodiment H. The composite article according to any of the previous Embodiments wherein in the portion of the strip bonded to the layer, the hooks have been crushed.

Embodiment I. The composite article according to Embodiments A through G wherein in the area of the strip bonded to the layer, the strip lacks hooks.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

FIG. 1A is a schematic plan view of a disposable diaper according to the background art.

FIG. 1B is a schematic side view of a detail 1B in FIG. 1A.

FIG. 2A is a schematic plan view of an embodiment of a disposable diaper according to the present disclosure.

FIG. 2B is a schematic side view of a detail 2B in FIG. 2A.

FIG. 3 is a schematic side detail view similar to detail FIG. 2B, illustrating an alternate embodiment according to the present disclosure.

FIG. 4 is a schematic side detail view similar to detail FIG. 2B, illustrating an alternate embodiment according to the present disclosure.

FIG. 5 is a schematic side detail view similar to detail FIG. 2B, illustrating an alternate embodiment according to the present disclosure.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure describes a garment such as a disposable diaper having a hook tab providing good strength while offering good flexibility and low cost. In some convenient embodiments, the tab also has loop material on its reverse side, allowing more flexibility when worn by persons at the lower end of the size range for whom the garment is intended.

For the following Glossary of defined terms, these definitions shall be applied for the entire application, unless a different definition is provided in the claims or elsewhere in the specification.

### Glossary

Certain terms are used throughout the description and the claims that, while for the most part are well known, may require some explanation. It should understood that, as used herein:

The terms “(co)polymer” or “(co)polymers” includes homopolymers and copolymers, as well as homopolymers or copolymers that may be formed in a miscible blend, e.g., by coextrusion or by reaction, including, e.g., transesterification. The term “copolymer” includes random, block and star (e.g. dendritic) copolymers.

The term “adjoining” with reference to a particular layer means joined with or attached to another layer, in a position wherein the two layers are either next to (i.e., adjacent to) and directly contacting each other, or contiguous with each other but not in direct contact (i.e., there are one or more additional layers intervening between the layers). By using terms of orientation such as “atop”, “on”, “over,” “covering”, “uppermost”, “underlying” and the like for the location of various elements in the disclosed coated articles, we refer to the relative position of an element with respect to a horizontally-disposed, upwardly-facing substrate. However, unless otherwise indicated, it is not intended that the substrate or articles should have any particular orientation in space during or after manufacture.

The terms “about” or “approximately” with reference to a numerical value or a shape means +/−five percent of the numerical value or property or characteristic, but expressly includes the exact numerical value. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

As used in this specification, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Exemplary Diaper Embodiments

Various exemplary embodiments of the disclosure will now be described with particular reference to the Drawings.

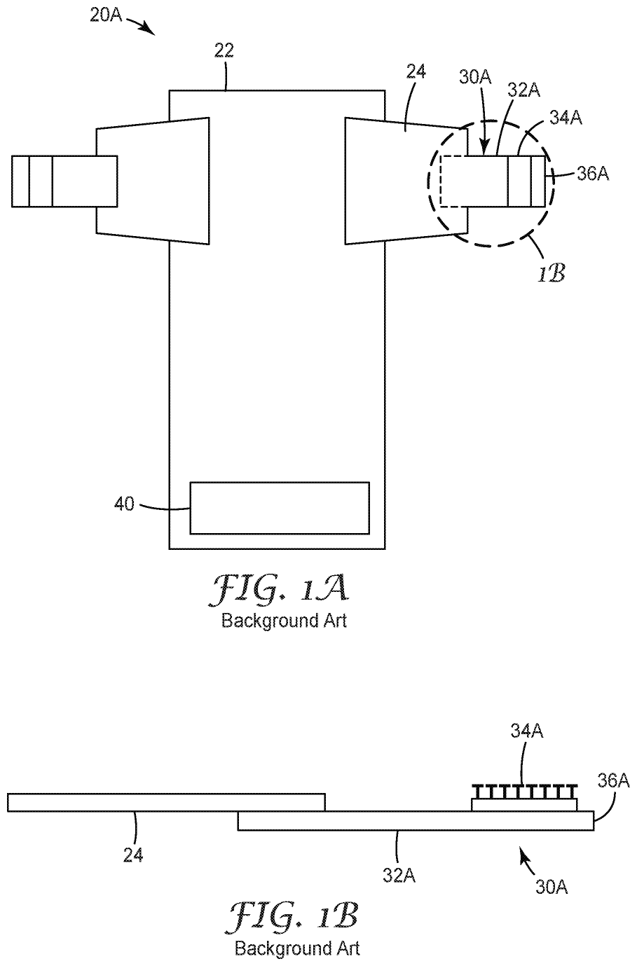

Referring now to FIG. 1A, a schematic plan view of a disposable diaper 20A according to the background art is illustrated. Disposable diaper 20A includes a chassis 22 of conventional type, typically a composite structure with an absorbent material included. A pair of ears 24 extend laterally from chassis 22. In many known embodiments, ears 24 are attached to chassis 22 by adhesive bonding and/or thermal bonding and/or pressure bonding rather than being integral to chassis 22 so as to minimize cutting waste of the material of chassis 22. Attached to ears 24 are tabs 30A, which include a ribbon 32A and a strip of hook material 34A. Ribbon 32A may have a tab 36A which extends laterally beyond the edge of hook material 34A to aid in removing diaper 20A after use. When diapering the user of diaper 20A, hook material 34A assumes a position opposite a landing zone 40 of loop material, and is secured to it by hook-and-loop engagement.

Referring now to FIG. 1B, a schematic side view of a detail 1B in FIG. 1A is illustrated. In this view, the arrangement of the layers can be more easily visualized. It will be noted that an alternate arrangement with ear 24 on the opposite side of ribbon 32A from hook material 34A is also visible.

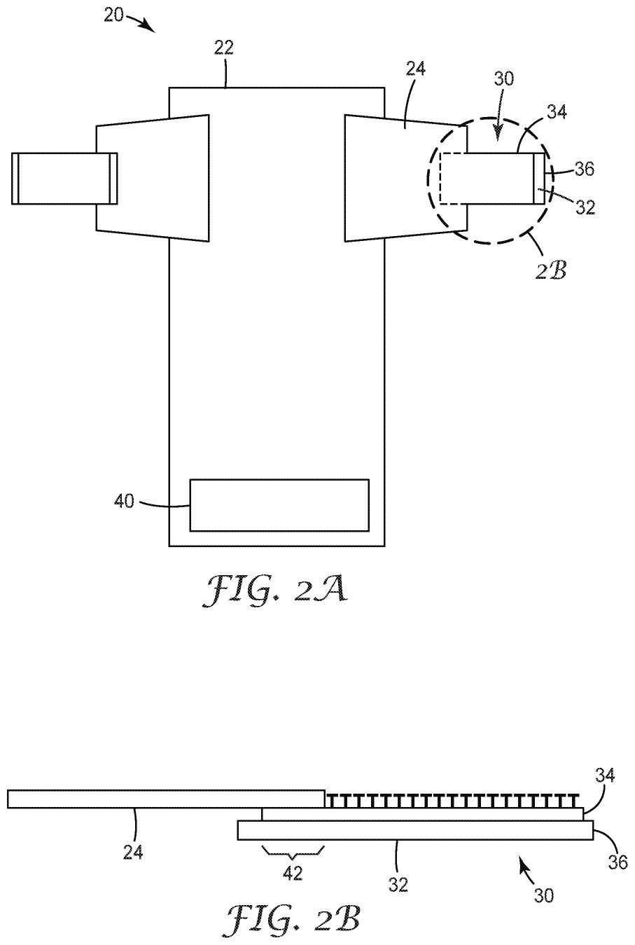

Referring now to FIG. 2A, a schematic plan view of an embodiment of a disposable diaper 20 according to the present disclosure is illustrated. While visually similar to the disposable diaper 20A of the background art illustrated in FIG. 1A, an important difference allows tab 30 to be reinforced providing an advantage of superior strength. Disposable diaper 20 includes a chassis 22 of conventional type, typically a composite structure with an absorbent material included. A pair of ears 24 extend laterally from chassis 22. In the illustrated embodiment, ears 24 are attached to chassis 22 by adhesive bonding and/or thermal bonding and/or pressure bonding rather than being integral to chassis 22 so as to minimize cutting waste of the material of chassis 22. However, the present disclosure also contemplates embodiments where the ears are integral. Attached to ear 24 are the proximal end of a tab 30, which include a ribbon 32 and a strip of hook material 34. The distal end of ribbon 32 may have a tab 36 free of hooks which extends laterally beyond the edge of hook material 34 to aid in removing diaper 20 after use.

When diapering the user of diaper 20, hook material 34 assumes a position opposite a landing zone 40 of loop material, and is secured to it by hook-and-loop engagement. In this embodiment, however, the hook material extends farther along ribbon 32, so that it is hook material 34 that partly or wholly contacts ear 24.

Referring now to FIG. 2B, a schematic side view of a detail 2B in FIG. 2A is illustrated. In this view, the arrangement of the layers can be more easily visualized. In this view, it can be appreciated how hook material 24 extends so as to be in contact with ear 24. In forming this embodiment, the portion 42 of hook material 34 underlying ear 24 may have its hooks deliberately crushed in a separate operation before hook material 34 is bonded by, e.g., heat, ultrasonic energy, or adhesive to ear 24. Alternatively, the hook may be left uncrushed and simply be destroyed or subsumed in the bonding operation.

Referring now to FIG. 3, a schematic side detail view similar to detail FIG. 2B, but illustrating an alternate embodiment according to the present disclosure is presented. In this embodiment, ribbon 32 has been omitted.

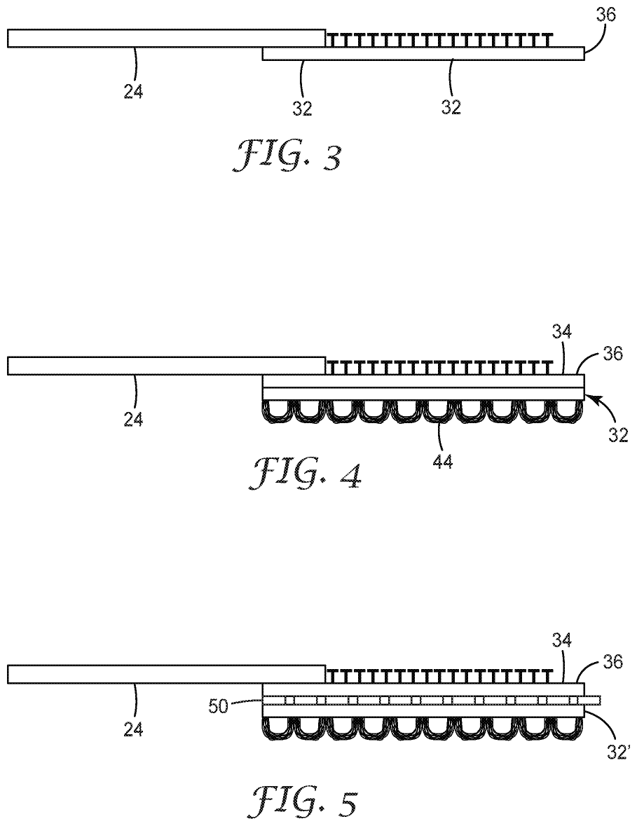

Referring now to FIG. 4, a schematic side detail view similar to detail FIG. 2B, but illustrating an alternate embodiment according to the present disclosure is presented. In this embodiment, ribbon 32′, which includes loop structures 44, has been substituted for ribbon 32 as in, e.g., FIG. 2B. This allows the two tabs on either side of diaper 20 (as in FIG. 2A) to attach in a hook-and-loop fashion to each other as an alternative to them attaching to landing zone 40. This allows the diaper to be conveniently worn by a person on the low end of the size range for which the diaper is intended.

Referring now to FIG. 5, a schematic side detail view similar to detail FIG. 4, but illustrating an alternate embodiment according to the present disclosure is presented. In this embodiment, a layer 50 comprising indicia is present, as a separate layer, or a printed upon either hook material 34 or ribbon 32′. In this embodiment, either hook material 34 or ribbon 32′ is formed transparent so that the indicia on layer 50 can be seen by the user.

The operation of exemplary embodiments of the present disclosure will be further described with regard to the following non-limiting detailed Examples. These examples are offered to further illustrate the various specific and preferred embodiments and techniques. It should be understood, however, that many variations and modifications may be made while remaining within the scope of the present disclosure.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Comparative Example C-1

A tab was prepared generally similar to that depicted in FIG. 1B. More specifically, a roll of polypropylene hook material commercially available as HV-Series Hook, CHK-05810 from 3M Company, of St. Paul, Minn., was slit down to 13 mm widths. These widths were then unwound and cut into segments 25 mm long. A roll of support fabric, comprising polypropylene film having polypropylene fibers imbedded therein commercially available as EBL LIGHT from 3M Company was slit down to a width of 60 mm. It was then unwound and cut into a 25 mm segments giving each a width of 60 mm and a width 25 mm. The strip material was then laminated to the support fabric such that the 25 mm length was across the 25 mm width of the support fabric. The edge of the hook material nearest the distal end of the support fabric was spaced 5 mm from the distal end of the support fabric. The lamination was accomplished using an acrylate transfer adhesive provided on a fiber filled polyester backing, commercially available as Medical Transfer Adhesive 1524 from 3M Company, giving a final sample size of 60 mm by 25 mm.

This sample was put into tension test apparatus commercially available as Model Number 5564 from Instron of Norwood, Mass. so as to measure the combined tensile strength of the strip plus the support fabric. The jaws of the Instron were spaced apart by 30 mm. The jaws started from an initial condition where they overlapped and clamped onto the distal and proximal ends of the sample by 15 mm from each end. Tensile stress was applied with the jaws separating at a rate of 500 mm/min. The tension at failure as an average of four runs was 6.79 N/cm of width.

### Example 1

A tab was prepared generally similar to that depicted in FIG. 3. More specifically, a roll of hook material commercially available as HV-Series Hook, CHK-05810 was slit down to a width of 60 mm. The roll of strip material was slit down to a width of 60 mm. It was then unwound and cut into a 25 mm segment giving it a width of 60 mm and a length of 25 mm. The basis weight of the strip was less than 80 g/m².

This sample was put into the Instron tester to measure tensile strength of the strip. The jaws of the Instron were spaced apart by 30 mm. The jaws overlapped and clamped onto the proximal edge of the strip by 15 mm to simulate the attachment zone where the strip is bonded to the layer. The jaws overlapped and clamped onto the distal edge of the strip by 15 mm to simulate a finger lift. The tension at failure as an average of four runs was 11.84 N/cm of width.

### Example 2

A tab was prepared generally similar to that depicted in FIG. 4. More specifically, a roll of hook material commercially available as HV-Series Hook, CHK-05810 was slit down to a width of 60 mm. It was then unwound and cut into a 25 mm long segment giving it a width of 60 mm and a length of 25 mm. A roll of support fabric comprising EBL LIGHT was slit down to a width of 60 mm. It was then unwound and cut into a 25 mm segment giving it a width of 60 mm and a length of 25 mm. The strip material was then laminated to the support fabric using 3M Medical transfer adhesive 1524 giving a final sample size of 60 mm by 25 mm.

This sample was put into the Instron tester to measure tensile strength of the strip. The jaws of the Instron were spaced apart by 30 mm. The jaws overlapped and clamped onto the proximal edge of the strip by 15 mm to simulate the attachment zone where the strip is bonded to the layer. The jaws overlapped and clamped onto the distal edge of the strip by 15 mm to simulate a finger lift. The tension at failure as an average of two runs was 18.62 N/cm of width.

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. In particular, as used herein, the recitation of numerical ranges by endpoints is intended to include all numbers subsumed within that range (e.g., 1 to 5 includes 1, 1.5, 2, 2.75, 3, 3.80, 4, and 5). In addition, all numbers used herein are assumed to be modified by the term “about.”

Furthermore, all publications and patents referenced herein are incorporated by reference in their entirety to the same extent as if each individual publication or patent was specifically and individually indicated to be incorporated by reference. Various exemplary embodiments have been described. These and other embodiments are within the scope of the following claims.

1. A composite article comprising a layer and a strip having raised hooks on a first side, wherein a portion of the first side is bonded to the layer.

2. The composite article according to claim 1 wherein the layer is a portion of a disposable diaper.

3. The composite article according to claim 1, wherein the strip has a second side opposite the first side, and wherein the second side is bonded to a support fabric.

4. The composite article according to claim 1, wherein the support fabric comprises loop material.

5. The composite article according to claim 1, wherein the strip is bonded to the layer at a proximal end of the strip, and wherein a tab at the distal end of the strip is free of raised hooks.

6. The composite article according to claim 5 wherein the distal end has flattened hooks.

7. The composite article according to claim 1 wherein the basis weight of the strip is less than 80 g/m².

8. The composite article according to claim 1 wherein in the area of the strip bonded to the layer, the hooks have been crushed.

9. The composite article according to claim 1 wherein in the area of the strip bonded to the layer, the strip lacks hooks.
